(19) United States
(12) Patent Application Publication
(10) Pub. No.: US 2017/0348388 A1
(43) Pub. Date: Dec. 7, 2017

(54) ADJUVANTS USEFUL FOR STIMULATION OF IMMUNITY TO TUMOR ENDOTHELIAL CELLS

(71) Applicant: Batu Biologics, Inc., San Diego, CA (US)

(72) Inventors: Samuel C. Wagner, San Diego, CA (US); Thomas E. Ichim, San Diego, CA (US)

(73) Assignee: Batu Biologics, Inc., San Diego, CA (US)

(21) Appl. No.: 15/611,694

(22) Filed: Jun. 1, 2017

Related U.S. Application Data
(60) Provisional application No. 62/344,879, filed on Jun. 2, 2016.

(51) Int. Cl. A61K 38/19; A61K 39/395; A61K 39/00; A61K 39/21; A61K 48/00

(52) U.S. Cl. CPC A61K 38/19; A61K 39/0011; A61K 39/21; A61K 39/395; A61K 39/00; A61K 2039/5152; A61K 2039/5156; A61K 2039/55522; A61K 48/00

(57) ABSTRACT

Adjuvants acting at the level of antigen presentation useful for stimulation of specific immunity against tumor endothelial cells. In one embodiment the invention teaches the use of activation of specific antigen presenting cell programs to selectively induce cytotoxic T cells and antibodies with complement activating ability while not evoking antigenic responses that potentially stimulate angiogenesis or growth of tumor endothelial cells. Specifically, the invention teaches selection and use of adjuvants that inhibit suppressive dendritic cell produced factors, surface bound and soluble, while stimulating activatory cytokines. Adjuvant means include innate activatory molecules, gene silencing means, alarmins, and other stimulatory means resembling “danger” signals.

### CROSS-REFERENCE TO RELATED APPLICATION

This application claims the benefit of U.S. Provisional Application No. 62/344,879 filed on Jun. 2, 2016, entitled “ADJUVANTS USEFUL FOR STIMULATION OF IMMUNITY TO TUMOR ENDOTHELIAL CELLS”, the contents of which are incorporated herein by reference as though set forth in their entirety.

### FIELD OF THE INVENTION

The invention pertains to the field of immunology, more specifically, the invention pertains to the use of the immune system to target cancers in a mammal, more specifically, the invention pertains to the targeting of cancer blood vessels using the immune system of said mammal in order to selectively cause death of said tumor while sparing neoplastic-associated vasculature, more specifically, the invention relates to programming of the immune system to selectively induce immune responses that provide only tumor neoangiogenesis ablative activity while not stimulating immunity capable of promoting neovasculature associated with neoplasia.

### BACKGROUND OF THE INVENTION

Treatment of cancer using immunotherapy has been described as “Breakthrough of the Year” in light of positive data generated utilizing checkpoint inhibitors, as well as chimeric antigen receptor (CAR) T cells. Unfortunately, despite promising effects of current clinical protocols, response rates still are between 10-30%, with some tumor types not responding.

The utilization of antigen-specific immune stimulation is potentially superior to antigen-nonspecific approaches such as checkpoint inhibitors. When checkpoint inhibitors are used clinically, latent T cell clones are activated to proliferate. While this includes tumor specific T cells, that are generally repressed by tumors, this also includes autoreactive T cells. This explains the higher incidence of toxicities associated with autoimmunity in patients receiving checkpoint inhibitors. It has been reported that up to 20% of patients receiving checkpoint inhibitors have some degree of autoimmunity, most prevalently colitis. Given the recent introduction of checkpoint inhibitors into widespread clinical use, it may be that autoimmunity may develop in cancer patients during analysis of extended follow-up.

While it is intellectually appealing to augment cancer specific, or cancer endothelial specific clones with vaccination, a draw-back of cancer vaccination is the potential to augment or accelerate tumor growth in response to the vaccine. For example, Flexner and Jobling, showed that injection of dead autologous tumor cells enhanced the growth of pre-existing tumors. In general, Th2-driven antibody responses to tumors are non-protective and may contribute to tumor progression by inhibiting the Th1 cell-mediated immune response. It may be that this occurs because of non-useful adjuvants being administered that stimulate Th2 responses as compared to Th1, which are known to induce cytotoxic antibodies. Kaliss popularized the term “immunological enhancement” to describe the enhancement of tumor growth by non-cytotoxic antibodies. It was theorized that these antibodies bind to tumor cells, masking their epitopes and thus preventing a cell-mediated immune response, although this has never been demonstrated experimentally. This is similar to the theory of immunostimulation of tumor growth, which states that, in contrast to the strong immune response generated by transplantable tumors, a quantitatively mild immune response, such as that generated by spontaneous tumors, is stimulatory to the growth of neoplasia. Several experimental observations support the hypothesis that such a weak immune response to cancer may stimulate tumor growth. The co-injection of lymphocytes (spleen cells) from syngeneic mice that had been growing tumors for 10-20 days with tumor cells from MCA-induced sarcomas into thymectomized irradiated syngeneic mice at a range of doses accelerated tumor growth when the ratio of lymphocytes to tumor cells was low. However, when the ratio of lymphocytes to tumor cells was high, lymphocytes from specifically immunized mice inhibited growth compared with naïve lymphocytes that continued to augment tumor growth. This suggests the existence of a biphasic dose response whereas a “weak” immune response results in stimulation of tumor growth while a strong immune response results in protection. One evidence for enhancement of tumor growth in response to vaccination is provided by cancer vaccine clinical trials in which vaccination augments tumor relapse. Thus there is a need in the field to augment means of stimulating immune responses that selectively kill tumor cells and/or tumor endothelial cells without stimulating augmentation of cancer relapse/metastasis.

### SUMMARY OF THE INVENTION

A method of stimulating an antibody mediated cytotoxic response to tumor endothelium may comprise the steps: a) selecting an antigen or antigenic composition that is preferentially expressed on tumor endothelial cells; b) administering the antigen or antigenic composition together with an adjuvant or plurality of adjuvants capable of stimulating a cytotoxic antibody mediated response; c) assessing ability of generated antibody to evoke angiogenesis versus induce killing of tumor endothelial cells; and d) modify frequency, route of administration, or adjuvant type to enhance specific killing of tumor endothelial cells while reducing stimulation of angiogenesis. The antigen expressed on tumor endothelial cells may be selected from a group comprising: a) ROBO-4; b) VEGF-R2; c) FGF-R; d) CD105; e) TEM-1; f) survivin; g) CD93; h) CD 109; and i) ROBO 1-18. The antigen expressed on tumor endothelial cells may be a plurality of antigens. The plurality of antigens are isolated from an endothelial cell source that has been generated under conditions representing the tumor microenvironment. The conditions representing the tumor microenvironment may be increased acidity compared to non-malignant tissues. The conditions representing the tumor microenvironment may be increased hypoxia compared to non-malignant tissues. The conditions representing the tumor microenvironment may be increased angiogenic factors compared to non-malignant tissues. The angiogenic factors are selected form a group comprising of: a) VEGF; b) FGF-1; c) FGF-2; d) FGF-5; e) TGF-beta; f) EGF; g) HGF; h) IGF; i) PDGF-BB; j) placental protein-14; and k) angiopoietin. The conditions representing the tumor microenvironment may be enhanced expression of soluble immune suppressive molecules compared to non-malignant tissues. The immune suppressive molecule may be IL-10. The immune suppressive molecule may be IL-6. The immune suppressive molecule may be PGE-2. The immune suppressive molecule may be a tryptophan metabolite. The tryptophan metabolite may be kynurenine. The tryptophan metabolite may be putriscine. The tryptophan metabolite may be spermine. The immune suppressive molecule may be an arginine metabolite. The arginine metabolite may be ornithine. The arginine metabolite may be a polyamine. The endothelial cells may be a placental derived endothelial cell.

### DETAILED DESCRIPTION OF THE INVENTION

The invention teaches uses of specific adjuvants in order to augment specific immune responses associated with inhibition of endothelial proliferation while concurrently suppressing generation of antibodies that are capable of enhancing tumor growth. In one embodiment, compounds that activate DC are utilized as adjuvants with the idea of selectively stimulating Th1 responses. Once DC are activated by a stimulatory signal such as a TLR agonist, phagocytic activity decreases and the DC then migrate into the draining lymph nodes through the afferent lymphatics. During the trafficking process, DC degrade ingested proteins into peptides that bind to both MHC class I molecules and MHC class II molecules. This allows the DC to: a) perform cross presentation in that they ingest exogenous antigens but present peptides in the MHC I pathway; and b) activate both CD8 (via MHC I) and CD4 (via MHC II). Interestingly, lipid antigens are processed via different pathways and are loaded onto non-classical MHC molecules of the CD1 family. In one embodiment of the invention DC are cultured with placental endothelial cells that have been treated in a manner to render said placental endothelial cells to resemble cancer endothelial cells. Properties of cancer endothelial cells are known in the art and include expression of TEM-1. In one embodiment TEM-1 is used as a cancer endothelium specific antigen for pulsing of dendritic cells. TEM-1 is one of several proteins that are localized to the tumor stromal compartment. The protein was first discovered using a whole cell immune approach, whereby human fetal fibroblasts that have many characteristics similar to stromal cell fibroblasts, were used to immunize immunocompetent mice. These efforts led to the development of an antibody called FB5 that recognized an antigen associated with tumor stroma. Years later, an independent effort identified cell surface markers on primary tumor endothelium via Serial Analysis of Gene Expression (SAGE). This research identified the TEM-1 gene product as the FB5 antigen. Further examination of gene expression patterns in normal and neoplastic tissue have indicated up-regulation of TEM-1 expression in tumor neovessels within numerous histologically distinct tumor tissues.

The possibility of using dendritic cells to act as antigen presenting cells for ValloVax, or other tumor endothelial targeting antigenic sources can be realized by adapting techniques routinely used in the context of killing of tumors. Numerous animal models have demonstrated that in the context of neoplasia DCs can bind to and engulf tumor antigens that are released from tumor cells, either alive or dying, and cross-present these antigens to T cells in tumor-draining lymph nodes. This results in the generation of tumor-specific immune responses that have been demonstrated to inhibit tumor growth or in some cases induced transferrable immunological memory. Mechanistically, DCs recognize tumors using the same molecular means that they would use to recognize apoptotic cells, or cells that are stressed. One set of signals are molecules released from apoptotic cells, which are copiously released by tumors, these include the nucleotides UTP and ATP, fractalkine, lipid lysophosphatidylcholine, and sphingosine 1-phosphate. Signals from stressed cells, such as tumor cells include externalization of phosphatidylserine onto the outside of the cell membrane, calreticulin, avB5 integrin, CD36 and lactadherin. There is some evidence that dendritic cells actively promote tumor immunogenicity in that patients with dendritic cell infiltration of tumors generally have a better prognosis.

In one embodiment the invention teaches the use of adjuvants that modulate dendritic cells to stimulate antibodies that are cytotoxic, for example, complement-fixing. In one embodiment, tumor endothelial antigens are co-administered together with adjuvants that stimulate dendritic cells to program T cells in a manner to allow T cell upregulation of cytokines associated with cytotoxic antibodies, such as interferon gamma, or BLyS. In one embodiment adjuvants are selected from a group comprising of: Cationic liposome-DNA complex JVRS-100, aluminum hydroxide, aluminum phosphate vaccine, aluminum potassium sulfate adjuvant, Alhydrogel, ISCOM(s), Freund's Complete Adjuvant, Freund's Incomplete Adjuvant, CpG DNA Vaccine Adjuvant, Cholera toxin, Cholera toxin B subunit liposomes, Saponin, DDA, Squalene-based Adjuvants, Etx B subunit, IL-12, LTK63 Vaccine Mutant Adjuvant, TiterMax Gold Adjuvant, Ribi Vaccine Adjuvant, Montanide ISA 720 Adjuvant, Corynebacterium-derived P40 Vaccine Adjuvant, MPL™ Adjuvant, AS04, AS02, Lipopolysaccharide Vaccine Adjuvant, Muramyl Dipeptide Adjuvant, CRL1005, Killed Corynebacterium parvum Vaccine Adjuvant, Montanide ISA 51, Bordetella pertussis component Vaccine Adjuvant, Cationic Liposomal Vaccine Adjuvant, Adamantylamide Dipeptide Vaccine Adjuvant, Arlacel A, VSA-3 Adjuvant, Aluminum vaccine adjuvant, Polygen Vaccine Adjuvant, Adjumer™, Algal Glucan, Bay R1005, Theramide®, thalidomide, Stearyl Tyrosine, Specol, Algammulin, Avridine®, Calcium Phosphate Gel, CTA1-DD gene fusion protein, DOC/Alum Complex, Gamma Inulin, Gerbu Adjuvant, GM-CSF, GMDP, Recombinant hIFN-gamma/Interferon-g, Interleukin-1β, Interleukin-2, Interleukin-7, Sclavo peptide, Rehydragel LV, Rehydragel HPA, Loxoribine, MF59, MTP-PE Liposomes, Murametide, Murapalmitine, D-Murapalmitine, NAGO, Non-Ionic Surfactant Vesicles, PMMA, Protein Cochleates, QS-21, SPT (Antigen Formulation), nanoemulsion vaccine adjuvant, AS03, Quil-A vaccine adjuvant, RC529 vaccine adjuvant, LTR192G Vaccine Adjuvant, E. coli heat-labile toxin, LT, amorphous aluminum hydroxyphosphate sulfate adjuvant, Calcium phosphate vaccine adjuvant, Montanide Incomplete Seppic Adjuvant, Imiquimod, Resiquimod, AF03, Flagellin, Poly(I:C), ISCOMATRIX®, Abisco-100 vaccine adjuvant, Albumin-heparin microparticles vaccine adjuvant, AS-2 vaccine adjuvant, B7-2 vaccine adjuvant, DHEA vaccine adjuvant, Immunoliposomes Containing Antibodies to Costimulatory Molecules, SAF-1, Sendai Proteoliposomes, Sendai-containing Lipid Matrices, Threonyl muramyl dipeptide (TMDP), Ty Particles vaccine adjuvant, Bupivacaine vaccine adjuvant, DL-PGL (Polyester poly (DL-lactide-co-glycolide)) vaccine adjuvant, IL-15 vaccine adjuvant, LTK72 vaccine adjuvant, MPL-SE vaccine adjuvant, non-toxic mutant E112K of Cholera Toxin mCT-E112K, and Matrix-S.

For the purpose of the invention, several terms utilized in the art are defined:

“Adjuvant” refers to a substance that is capable of enhancing, accelerating, or prolonging an immune response when given with a vaccine immunogen.

“Agonist” refers to is a substance which promotes (induces, causes, enhances or increases) the activity of another molecule or a receptor. The term agonist encompasses substances which bind receptor (e.g., an antibody, a homolog of a natural ligand from another species) and substances which promote receptor function without binding thereto (e.g., by activating an associated protein).

“Antagonist” or “inhibitor” refers to a substance that partially or fully blocks, inhibits, or neutralizes a biological activity of another molecule or receptor.

“Co-administration” refers to administration of two or more agents to the same subject during a treatment period. The two or more agents may be encompassed in a single formulation and thus be administered simultaneously. Alternatively, the two or more agents may be in separate physical formulations and administered separately, either sequentially or simultaneously to the subject. The term “administered simultaneously” or “simultaneous administration” means that the administration of the first agent and that of a second agent overlap in time with each other, while the term “administered sequentially” or “sequential administration” means that the administration of the first agent and that of a second agent does not overlap in time with each other.

“Immune response” refers to any detectable response to a particular substance (such as an antigen or immunogen) by the immune system of a host vertebrate animal, including, but not limited to, innate immune responses (e.g., activation of Toll receptor signaling cascade), cell-mediated immune responses (e.g., responses mediated by T cells, such as antigen-specific T cells, and non-specific cells of the immune system), and humoral immune responses (e.g., responses mediated by B cells, such as generation and secretion of antibodies into the plasma, lymph, and/or tissue fluids). Examples of immune responses include an alteration (e.g., increase) in Toll-like receptor activation, lymphokine (e.g., cytokine (e.g., Th1, Th2 or Th17 type cytokines) or chemokine) expression or secretion, macrophage activation, dendritic cell activation, T cell (e.g., CD4+ or CD8+T cell) activation, NK cell activation, B cell activation (e.g., antibody generation and/or secretion), binding of an immunogen (e.g., antigen (e.g., immunogenic polypolypeptide)) to an MHC molecule, induction of a cytotoxic T lymphocyte (“CTL”) response, induction of a B cell response (e.g., antibody production), and, expansion (e.g., growth of a population of cells) of cells of the immune system (e.g., T cells and B cells), and increased processing and presentation of antigen by antigen presenting cells. The term “immune response” also encompasses any detectable response to a particular substance (such as an antigen or immunogen) by one or more components of the immune system of a vertebrate animal in vitro.

“Treating a cancer”, “inhibiting cancer”, “reducing cancer growth” refers to inhibiting or preventing oncogenic activity of cancer cells. Oncogenic activity can comprise inhibiting migration, invasion, drug resistance, cell survival, anchorage-independent growth, non-responsiveness to cell death signals, angiogenesis, or combinations thereof of the cancer cells. The terms “cancer”, “cancer cell”, “tumor”, and “tumor cell” are used interchangeably herein and refer generally to a group of diseases characterized by uncontrolled, abnormal growth of cells (e.g., a neoplasioa). In some forms of cancer, the cancer cells can spread locally or through the bloodstream and lymphatic system to other parts of the body (“metastatic cancer”). “Ex vivo activated lymphocytes”, “lymphocytes with enhanced antitumor activity” and “dendritic cell cytokine induced killers” are terms used interchangeably to refer to composition of cells that have been activated ex vivo and subsequently reintroduced within the context of the current invention. Although the word “lymphocyte” is used, this also includes heterogenous cells that have been expanded during the ex vivo culturing process including dendritic cells, NKT cells, gamma delta T cells, and various other innate and adaptive immune cells. As used herein, “cancer” refers to all types of cancer or neoplasm or malignant tumors found in animals, including leukemias, carcinomas and sarcomas. Examples of cancers are cancer of the brain, melanoma, bladder, breast, cervix, colon, head and neck, kidney, lung, non-small cell lung, mesothelioma, ovary, prostate, sarcoma, stomach, uterus and Medulloblastoma.

In one embodiment of the invention, immunization against antigens found on tumor endothelium, is induced utilizing a primary immunization utilizing a polyvalent combination together with a suitable adjuvant. In one embodiment, said adjuvant is capable of inducing DC maturation in vivo. In a preferred embodiment, administration of tumor endothelial antigens is performed in combination with a mixture of autologous lymphocytice lysates. Said lysates are selected based on molecular weight and/or charge based on ability to stimulate dendritic cells in vitro. Stimulation of dendritic cells is quantified by upregulation of expression of costimulatory molecules on said dendritic cells, whereas said costimulatory molecules may be membrane bound, such as CD80, CD86 and CD40, or may be soluble signals such as IL-12.

Subsequent to immunization, specific immunity in a personalized manner is assessed to determine whether said antibodies are specific towards stimulation of complement dependent cytotoxicity, versus stimulation of growth factors production.

Immunity may be assessed to specific antigens found on tumor endothelial cells, antigens include ROBO, VEGFR, VEGFR2, FGFR, TEM-1 and notch. Assessment of immunity is performed by quantifying reactivity of T cells or B cells in response to protein antigens or derivatives thereof, derivatives including peptide antigens or other antigenic epitopes. Responses may be assessed in terms of proliferative responses, cytokine release, antibody responses, or generation of cytotoxic T cells. Methods of assessing said responses are well known in the art. In a preferred embodiment, antibody responses are assessed to a panel of tumor endothelium associated proteins subsequent to immunization of patient. Antibody responses are utilized to guide which peptides will be utilized for prior immunization. For example, if a patient is immunized with ValloVax on a weekly basis, the subsequent assessment of antibody responses is performed at approximately 1-3 months after initiation of immunization. Protocols for immunization with ValloVax include weekly, biweekly, or monthly. Assessment of antibody responses is performed utilizing standard enzyme linked immunosorbent (ELISA) assay to provide a quantitative measurement, or alternatively binding with antigens in vitro to determine complement activating activity. Assessment of antibodies is performed, in one embodiment of the invention, against proteins associated with tumor endothelium such as ROBO, VEGFR, VEGFR2, FGFR, TEM-1 and notch. In patients in which a higher antibody responses is observed against VEGFR2, as an example, immunization with VEGFR2 antigenic epitopes is performed to enhance such specific immune response. In patients in which after ValloVax immunization possess an elevated antibody response to TEM-1, immunization with TEM-1 is performed. In patients in which antibodies significantly increase to numerous antigens, multiple peptide or antigenic combinations are utilized.

In one preferred embodiment, immunity to tumor antigens is linked to immunity to tumor endothelial antigens by co-immunization. Numerous tumor antigens can be utilized to amplify the immune response selectively, these can be chosen from known groups of tumor antigens such as ERG, WT1, ALS, BCR-ABL, Ras-mutant, MUC1, ETV6-AML, LMP2, p53 non-mutant, MYC-N, surviving, androgen receptor, RhoC, cyclin B1, EGFRvIII, EphA2, B cell or T cell idiotype, ML-IAP, BORIS, hTERT, PLAC1, HPV E6, HPV E7, OY-TES1, Her2/neu, PAX3, NY-BR-1, p53 mutant, MAGE A3, EpCAM, polysialic Acid, AFP, PAX5, NY-ESO1, sperm protein 17, GD3, Fucosyl GM1, mesothelin, PSMA, GD2, MAGE A1, sLe(x), HMWMAA, CYP1B1, sperm fibrous sheath protein, B7H3, TRP-2, AKAP-4, XAGE 1, CEA, Tn, GloboH, SSX2, RGS5, SART3, gp100, MelanA/MART1, Tyrosinase, GM3 ganglioside, Proteinase 3 (PR1), Page4, STn, Carbonic anhydrase IX, PSCA, Legumain, MAD-CT-1 (protamin2), PSA, Tie 2, MAD-CT2, PAP, PDGFR-beta, NA17, VEGFR2, FAP, LCK, Fos-related antigen, LCK, FAP.

Combination of polyvalent vaccines with other cellular therapies as the initial poly-immunogenic composition is envisioned within the context of the invention as an antigenic source for use with different adjuvants. In one embodiment cellular lysates of tumor cells, or tumor stem cells are loaded into dendritic cells. In one embodiment the invention provides a means of generating a population of cells with tumoricidal ability that are polyvalently reactive, to which focus is added by subsequent peptide specific vaccination. The generation of cytotoxic lymphocytes may be performed, in one embodiment by extracted 50 ml of peripheral blood from a cancer patient and peripheral blood monoclear cells (PBMC) are isolated using the Ficoll Method. PBMC are subsequently resuspended in 10 ml AIM-V media and allowed to adhere onto a plastic surface for 2-4 hours. The adherent cells are then cultured at 37° C. in AIM-V media supplemented with 1,000 U/mL granulocyte-monocyte colony-stimulating factor and 500 U/mL IL-4 after non-adherent cells are removed by gentle washing in Hanks Buffered Saline Solution (HBSS). Half of the volume of the GM-CSF and IL-4 supplemented media is changed every other day. Immature DCs are harvested on day 7. In one embodiment said generated DC are used to stimulate T cell and NK cell tumoricidal activity by pulsing with autologous tumor lysate. Specifically, generated DC may be further purified from culture through use of flow cytometry sorting or magnetic activated cell sorting (MACS), or may be utilized as a semi-pure population. DC pulsed with tumor lysate may be added into said patient in need of therapy with the concept of stimulating NK and T cell activity in vivo, or in another embodiment may be incubated in vitro with a population of cells containing T cells and/or NK cells. In one embodiment DC are exposed to agents capable of stimulating maturation in vitro and rendering them resistant to tumor derived inhibitory compounds such as arginase byproducts. Specific means of stimulating in vitro maturation include culturing DC or DC containing populations with a toll like receptor agonist. Another means of achieving DC maturation involves exposure of DC to TNF-alpha at a concentration of approximately 20 ng/mL. In order to activate T cells and/or NK cells in vitro, cells are cultured in media containing approximately 1000 IU/ml of interferon gamma. Incubation with interferon gamma may be performed for the period of 2 hours to the period of 7 days. Preferably, incubation is performed for approximately 24 hours, after which T cells and/or NK cells are stimulated via the CD3 and CD28 receptors. One means of accomplishing this is by addition of antibodies capable of activating these receptors. In one embodiment approximately, 2 ug/ml of anti-CD3 antibody is added, together with approximately 1 ug/ml anti-CD28. In order to promote survival of T cells and NK cells, was well as to stimulate proliferation, a T cell/NK mitogen may be used. In one embodiment the cytokine IL-2 is utilized. Specific concentrations of IL-2 useful for the practice of the invention are approximately 500 u/mL IL-2. Media containing IL-2 and antibodies may be changed every 48 hours for approximately 8-14 days. In one particular embodiment DC are included to said T cells and/or NK cells in order to endow cytotoxic activity towards tumor cells. In a particular embodiment, inhibitors of caspases are added in the culture so as to reduce rate of apoptosis of T cells and/or NK cells. Generated cells can be administered to a subject intradermally, intramuscularly, subcutaneously, intraperitoneally, intraarterially, intravenously (including a method performed by an indwelling catheter), intratumorally, or into an afferent lymph vessel. The immune response of the patient treated with these cytotoxic cells is assessed utilizing a variety of antigens found in tumor endothelial cells. When cytotoxic or antibody, or antibody associated with complement fixation are recognized to be upregulated in the cancer patient, subsequent immunizations are performed utilizing peptides to induce a focusing of the immune response.

In another embodiment DC are generated from leukocytes of patients by leukopheresis. Numerous means of leukopheresis are known in the art. In one example, a Frenius Device (Fresenius Com.Tec) is utilized with the use of the MNC program, at approximately 1500 rpm, and with a P1Y kit. The plasma pump flow rates are adjusted to approximately 50 mL/min. Various anticoagulants may be used, for example ACD-A. The Inlet/ACD Ratio may be ranged from approximately 10:1 to 16:1. In one embodiment approximately 150 mL of blood is processed. The leukopheresis product is subsequently used for initiation of dendritic cell culture. In order to generates a peripheral blood mononuclear cells from leukopheresis product, mononuclear cells are isolated by the Ficoll-Hypaque density gradient centrifugation. Monocytes are then enriched by the Percoll hyperosmotic density gradient centrifugation followed by two hours of adherence to the plate culture. Cells are then centrifuged at 500 g to separate the different cell populations. Adherent monocytes are cultured for 7 days in 6-well plates at 2×106 cells/mL RMPI medium with 1% penicillin/streptomycin, 2 mM L-glutamine, 10% of autologous, 50 ng/mL GM-CSF and 30 ng/mL IL-4. On day 6 immature dendritic cells are pulsed with tumor endothelial lysate or with ValloVax. Pulsing may be performed by incubation of lysates with dendritic cells, or may be generated by fusion of immature dendritic cells with ValloVax cells. Means of generating hybridomas or cellular fusion products are known in the art and include electrical pulse mediated fusion, or stimulation of cellular fusion by treatment with polyethelyne glycol. On day 7, the immature DCs are then induced to differentiate into mature DCs by culturing for 48 hours with 30 ng/mL interferon gamma (IFN-γ). During the course of generating DC for clinical purposes, microbiologic monitoring tests are performed at the beginning of the culture, on the fifth day and at the time of cell freezing for further use or prior to release of the dendritic cells. Administration of ValloVax pulsed dendritic cells is utilized as a polyvalent vaccine, whereas subsequent to administration antibody or t cell responses are assessed for induction of antigen specificity, peptides corresponding to immune response stimulated are used for further immunization to focus the immune response.

In some embodiments, culture of the immune effectors cells is performed after extracting from a patient that has been immunized with a polyvalent antigenic preparation. Specifically separating the cell population and cell sub-population containing a T cell can be performed, for example, by fractionation of a mononuclear cell fraction by density gradient centrifugation, or a separation means using the surface marker of the T cell as an index. Subsequently, isolation based on surface markers may be performed. Examples of the surface marker include CD3, CD8 and CD4, and separation methods depending on these surface markers are known in the art. For example, the step can be performed by mixing a carrier such as beads or a culturing container on which an anti-CD8 antibody has been immobilized, with a cell population containing a T cell, and recovering a CD8-positive T cell bound to the carrier. As the beads on which an anti-CD8 antibody has been immobilized, for example, CD8 MicroBeads), Dynabeads M450 CD8, and Eligix anti-CD8 mAb coated nickel particles can be suitably used. This is also the same as in implementation using CD4 as an index and, for example, CD4 MicroBeads, Dynabeads M-450 CD4 can also be used. In some embodiments of the invention, T regulatory cells are depleted before initiation of the culture. Depletion of T regulatory cells may be performed by negative selection by removing cells that express makers such as neuropilin, CD25, CD4, CTLA4, and membrane bound TGF-beta. Experimentation by one of skill in the art may be performed with different culture conditions in order to generate effector lymphocytes, or cytotoxic cells, that possess both maximal activity in terms of tumor killing, as well as migration to the site of the tumor. For example, the step of culturing the cell population and cell sub-population containing a T cell can be performed by selecting suitable known culturing conditions depending on the cell population. In addition, in the step of stimulating the cell population, known proteins and chemical ingredients, etc., may be added to the medium to perform culturing. For example, cytokines, chemokines or other ingredients may be added to the medium. Herein, the cytokine is not particularly limited as far as it can act on the T cell, and examples thereof include IL-2, IFN-.gamma., transforming growth factor (TGF)-.beta., IL-15, IL-7, IFN-.alpha., IL-12, CD40L, and IL-27. From the viewpoint of enhancing cellular immunity, particularly suitably, IL-2, IFN-.gamma., or IL-12 is used and, from the viewpoint of improvement in survival of a transferred T cell in vivo, IL-7, IL-15 or IL-21 is suitably used. In addition, the chemokine is not particularly limited as far as it acts on the T cell and exhibits migration activity, and examples thereof include RANTES, CCL21, MIP1.alpha., MIP1.beta., CCL19, CXCL12, IP-10 and MIG. The stimulation of the cell population can be performed by the presence of a ligand for a molecule present on the surface of the T cell, for example, CD3, CD28, or CD44 and/or an antibody to the molecule. Further, the cell population can be stimulated by contacting with other lymphocytes such as antigen presenting cells (dendritic cell) presenting a target peptide such as a peptide derived from a cancer antigen on the surface of a cell. In addition to assessing cytotoxicity and migration as end points, it is within the scope of the current invention to optimize the cellular product based on other means of assessing T cell activity, for example, the function enhancement of the T cell in the method of the present invention can be assessed at a plurality of time points before and after each step using a cytokine assay, an antigen-specific cell assay (tetramer assay), a proliferation assay, a cytolytic cell assay, or an in vivo delayed hypersensitivity test using a recombinant tumor-associated antigen or an immunogenic fragment or an antigen-derived peptide. Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring a T cell response such as a limiting dilution method. In vivo assessment of the efficacy of the generated cells using the invention may be assessed in a living body before first administration of the T cell with enhanced function of the present invention, or at various time points after initiation of treatment, using an antigen-specific cell assay, a proliferation assay, a cytolytic cell assay, or an in vivo delayed hypersensitivity test using a recombinant tumor-associated antigen or an immunogenic fragment or an antigen-derived peptide. Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring a T cell response such as a limiting dilution method. Further, an immune response can be assessed by a weight, diameter or malignant degree of a tumor possessed by a living body, or the survival rate or survival term of a subject or group of subjects. Said cells can be expanded in the presence of specific antigens associated with tumor endothelium and subsequently injected into the patient in need of treatment. Expansion with specific antigens includes coculture with proteins selected from a group comprising of: a) ROBO; b) VEGF-R2; c) FGF-R; d) CD105; e) TEM-1; and f) survivin. Other tumor endothelial specific or semi-specific antigens are known in the art that may be used.

Within the context of the invention, teachings are provided to amplify an antigen specific immune response following immunization with a polyvalent vaccine, in which the antigenic epitopes are used for immunization together with adjuvants such as toll like receptors (TLRs). These molecules are type 1 membrane receptors that are expressed on hematopoietic and non-hematopoietic cells. At least 11 members have been identified in the TLR family. These receptors are characterized by their capacity to recognize pathogen-associated molecular patterns (PAMP) expressed by pathogenic organisms. It has been found that triggering of TLR elicits profound inflammatory responses through enhanced cytokine production, chemokine receptor expression (CCR2, CCR5 and CCR7), and costimulatory molecule expression. As such, these receptors in the innate immune systems exert control over the polarity of the ensuing acquired immune response. Among the TLRs, TLR9 has been extensively investigated for its functions in immune responses. Stimulation of the TLR9 receptor directs antigen-presenting cells (APCs) towards priming potent, T_{H1}-dominated T-cell responses, by increasing the production of pro-inflammatory cytokines and the presentation of co-stimulatory molecules to T cells. CpG oligonucleotides, ligands for TLR9, were found to be a class of potent immunostimulatory factors. CpG therapy has been tested against a wide variety of tumor models in mice, and has consistently been shown to promote tumor inhibition or regression.

In some embodiments of the invention, specific antigens are immunized following polyvalent immunization, said specific antigens administered in the form of DNA vaccines. Numerous publications have reported animal and clinical efficacy of DNA vaccines which are incorporated by reference. In addition to direct DNA injection techniques, DNA vaccines can be administered by electroporation. The nucleic acid compositions, including the DNA vaccine compositions, may further comprise a pharmaceutically acceptable excipient. Examples of suitable pharmaceutically acceptable excipients for nucleic acid compositions, including DNA vaccine compositions, are well known to those skilled in the art and include sugars, etc. Such excipients may be aqueous or non aqueous solutions, suspensions, and emulsions. Examples of non-aqueous excipients include propylene glycol, polyethylene glycol, vegetable oils such as olive oil, and injectable organic esters such as ethyl oleate. Examples of aqueous excipient include water, alcoholic/aqueous solutions, emulsions or suspensions, including saline and buffered media. Suitable excipients also include agents that assist in cellular uptake of the polynucleotide molecule. Examples of such agents are (i) chemicals that modify cellular permeability, such as bupivacaine, (ii) liposomes or viral particles for encapsulation of the polynucleotide, or (iii) cationic lipids or silica, gold, or tungsten microparticles which associate themselves with the polynucleotides. Anionic and neutral liposomes are well-known in the art (see, e.g., Liposomes: A Practical Approach, RPC New Ed, IRL press (1990), for a detailed description of methods for making liposomes) and are useful for delivering a large range of products, including polynucleotides. Cationic lipids are also known in the art and are commonly used for gene delivery. Such lipids include Lipofectin™ also known as DOTMA (N—[I-(2,3-dioleyloxy) propyls N,N, N-trimethylammonium chloride), DOTAP (1,2-bis (oleyloxy)-3 (trimethylammonio) propane), DDAB (dimethyldioctadecyl-ammonium bromide), DOGS (dioctadecylamidologlycyl spermine) and cholesterol derivatives such as DCChol (3 beta-(N-(N′,N′-dimethyl aminomethane)-carbamoyl) cholesterol). A description of these cationic lipids can be found in EP 187,702, WO 90/11092, U.S. Pat. No. 5,283,185, WO 91/15501, WO 95/26356, and U.S. Pat. No. 5,527,928. A particular useful cationic lipid formulation that may be used with the nucleic vaccine provided by the disclosure is VAXFECTIN, which is a commixture of a cationic lipid (GAP-DMORIE) and a neutral phospholipid (DPyPE) which, when combined in an aqueous vehicle, self-assemble to form liposomes. Cationic lipids for gene delivery are preferably used in association with a neutral lipid such as DOPE (dioleyl phosphatidylethanolamine), as described in WO 90/11092 as an example. In addition, a DNA vaccine can also be formulated with a nonionic block copolymer such as CRL1005. Other immunization means include prime boost regiments. The polypeptide and nucleic acid compositions can be administered to an animal, including human, by a number of methods known in the art. Examples of suitable methods include: (1) intramuscular, intradermal, intraepidermal, intravenous, intraarterial, subcutaneous, or intraperitoneal administration, (2) oral administration, and (3) topical application (such as ocular, intranasal, and intravaginal application). One particular method of intradermal or intraepidermal administration of a nucleic acid vaccine composition that may be used is gene gun delivery using the Particle Mediated Epidermal Delivery (PMED™) vaccine delivery device marketed by PowderMed. PMED is a needle-free method of administering vaccines to animals or humans. The PMED system involves the precipitation of DNA onto microscopic gold particles that are then propelled by helium gas into the epidermis. The DNA-coated gold particles are delivered to the APCs and keratinocytes of the epidermis, and once inside the nuclei of these cells, the DNA elutes off the gold and becomes transcriptionally active, producing encoded protein. This protein is then presented by the APCs to the lymphocytes to induce a T-cell-mediated immune response. Another particular method for intramuscular administration of a nucleic acid vaccine provided by the present disclosure is electroporation. Electroporation uses controlled electrical pulses to create temporary pores in the cell membrane, which facilitates cellular uptake of the nucleic acid vaccine injected into the muscle. Where a CpG is used in combination with a nucleic acid vaccine, it is preferred that the CpG and nucleic acid vaccine are co-formulated in one formulation and the formulation is administered intramuscularly by electroporation. A helper T cell and cytotoxic T cell stimulatory polypeptide can be introduced into a mammalian host, including humans, linked to its own carrier or as a homopolymer or heteropolymer of active polypeptide units. Such a polymer can elicit increase immunological reaction and, where different polypeptides are used to make up the polymer, the additional ability to induce antibodies and/or T cells that react with different antigenic determinants of the tumor. Useful carriers known in the art include, for example, thyroglobulin, albumins such as human serum albumin, tetanus toxoid, polyamino acids such as poly(D-lysine:D-glutamic acid), influenza polypeptide, and the like. Adjuvants such as incomplete Freunds adjuvant, GM-CSF, aluminum phosphate, CpG containing DNA, inulin, Poly (IC), aluminum hydroxide, alum, or montanide can also be used in the administration of an helper T cell and cytotoxic T cell stimulatory polypeptide.

1. A method of stimulating an antibody mediated cytotoxic response to tumor endothelium, said method comprising the steps:
a) selecting an antigen or antigenic composition that is preferentially expressed on tumor endothelial cells;
b) administering said antigen or antigenic composition together with an adjuvant or plurality of adjuvants capable of stimulating a cytotoxic antibody mediated response;
c) assessing ability of generated antibody to evoke angiogenesis versus induce killing of tumor endothelial cells; and
d) modify frequency, route of administration, or adjuvant type to enhance specific killing of tumor endothelial cells while reducing stimulation of angiogenesis.

2. The method of claim 1, wherein said antigen expressed on tumor endothelial cells is selected from a group comprising:
a) ROBO-4;
b) VEGF-R2;
c) FGF-R;
d) CD105;
e) TEM-1;
f) survivin;
g) CD93;
h) CD 109; and
i) ROBO 1-18.

3. The method of claim 1, wherein said antigen expressed on tumor endothelial cells is a plurality of antigens.

4. The method of claim 3, wherein said plurality of antigens are isolated from an endothelial cell source that has been generated under conditions representing the tumor microenvironment.

5. The method of claim 4, wherein said conditions representing the tumor microenvironment is increased acidity compared to non-malignant tissues.

6. The method of claim 4, wherein said conditions representing the tumor microenvironment is increased hypoxia compared to non-malignant tissues.

7. The method of claim 4, wherein said conditions representing the tumor microenvironment is increased angiogenic factors comparted to non-malignant tissues.

8. The method of claim 7, wherein said angiogenic factors are selected form a group comprising:
a) VEGF;
b) FGF-1;
c) FGF-2;
d) FGF-5;
e) TGF-beta;
f) EGF;
g) HGF;
h) IGF;
i) PDGF-BB;
j) placental protein-14; and
k) angiopoietin.

9. The method of claim 4, wherein said conditions representing the tumor microenvironment is enhanced expression of soluble immune suppressive molecules compared to non-malignant tissues.

10. The method of claim 9, wherein said immune suppressive molecule is IL-10.

11. The method of claim 9, wherein said immune suppressive molecule is IL-6.

12. The method of claim 9, wherein said immune suppressive molecule is PGE-2.

13. The method of claim 9, wherein said immune suppressive molecule is a tryptophan metabolite.

14. The method of claim 13, wherein said tryptophan metabolite is kynurenine.

15. The method of claim 13, wherein said tryptophan metabolite is putriscine.

16. The method of claim 13, wherein said tryptophan metabolite is spermine.

17. The method of claim 9, wherein said immune suppressive molecule is an arginine metabolite.

18. The method of claim 17, wherein said arginine metabolite is ornithine.

19. The method of claim 17, wherein said arginine metabolite is a polyamine.

20. The method of claim 4, wherein said endothelial cells is a placental derived endothelial cell.
